(19) United States
(12) Patent Application Publication
(10) Pub. No.: US 2012/0059382 A1
(43) Pub. Date: Mar. 8, 2012

(54) GUIDE SYSTEMS AND METHODS FOR LIGAMENT RECONSTRUCTION

(76) Inventor: Lonnie E. PAULOS, Pensacola Beach, FL (US)

(21) Appl. No.: 13/292,062

(22) Filed: Nov. 8, 2011

Related U.S. Application Data
(63) Continuation-in-part of application No. 12/937,402, filed on Oct. 12, 2010.
(60) Provisional application No. 61/411,534, filed on Nov. 9, 2010.
(60) Provisional application No. 61/049,430, filed on Apr. 30, 2008.

(51) Int. Cl. A61B 17/90

(52) U.S. Cl. 606/96

(57) ABSTRACT

One embodiment of a ligament reconstruction guide assembly comprises a bracket, a cannulated guide and a reference element. Embodiments of these assembly elements are combined and shaped in a manner that allow a surgeon to carefully position the attachment sites for anterior or posterior cruciate ligament reconstruction procedures. Embodiments of the guide assembly comprise a footprint target tip that assists locating the attachment sites for single or double bundle reconstruction procedures from an anterior or posterior position. Embodiments of the guide assembly have the cannulated guide aligned with apertures in the footprint target tip to properly locate and position the bone tunnels. This combination and shape allows proper positioning of a drill and tunnel and reduces the possibility of wounds to the knee during tunnel creation. In some embodiments there are different shaped, removable footprint target tips.

### CROSS-REFERENCE TO RELATED APPLICATIONS

This application is a continuation in part of co-pending U.S. patent application Ser. No. 12/937,402, filed Oct. 12, 2010 which is the National Stage of International Application PCT App. No. PCT/US09/34988, filed Feb. 24, 2009, which claims the benefit of U.S. Pat. App. No. 61/049,430, filed on Apr. 30, 2008; this application also claims benefit of U.S. Pat. App. No. 61/411,534, filed Nov. 9, 2010; and the entire contents of all the above applications are incorporated herein by reference.

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF THE INVENTION

1. Technical Field

The present invention relates to an improved guide assembly to be used in the reconstruction of a ligament as well as methods for using the assembly. The present invention also relates to an improved ligament reconstruction guide assembly and methods for locating tibial and femoral bone tunnels in the reconstruction of anterior and posterior cruciate ligaments.

2. Background

Examples of guides to create tibial tunnels for anterior cruciate ligament (ACL) reconstruction include those described and disclosed in U.S. Pat. No. 6,254,605 to Howell, Stephen entitled “Tibial Guide” issued on Jul. 3, 2001 and U.S. Pat. No. 5,300,077 to Howell, Stephen entitled “Methods and instruments for ACL reconstruction” issued on Apr. 5, 1994 both of which are herein incorporated by reference in their entirety. These disclosures describe assemblies and methods for ACL bone tunnel creation from the front, anterior position of the knee.

Similarly, publication WO2007107697, published Sep. 27, 2007 for PCT application PCT/GB2007/000767 filed Mar. 5, 2007 and US publication US20090171355, published Jul. 2, 2009 for U.S. application Ser. No. 12/225,460 filed Mar. 5, 2008 to Andrew AMIS et al., both publications of which are incorporated by reference in their entirety, disclose a guide for ACL repair.

### BRIEF SUMMARY OF THE INVENTION

In one example embodiment of a guide assembly for locating a tunnel position on an attachment surface of a bone for a joint ligament reconstruction, the guide assembly comprises a reference element, a cannulated guide with at least one longitudinal bore having at least one longitudinal axis and the longitudinal axis being aligned relative to the reference element, a bracket connecting the reference element and the cannulated guide and the bracket having a means to align the longitudinal axis relative to the reference element positioned from a posterior position to a bone. In some embodiments, the means to provide the reference may be a footprint target tip to position an attachment site for single or double bundle ligament reconstruction procedure. In some embodiments, the means to provide the reference can be positioned from an anterior position to the bone.

In one example embodiment of a guide assembly for locating a tunnel position on an attachment surface of a bone for a joint ligament reconstruction, the guide assembly comprises a reference element having a footprint target tip, the footprint target tip having a plurality of apertures, at least one cannulated guide having a longitudinal bore defining a longitudinal bore axis, a bracket to align the longitudinal bore of the cannulated guide relative to the plurality of apertures and the bracket connecting the referent element and the cannulated guide whereby the reference element can be positioned from a posterior position to the bone. In some embodiments, the bracket is an adjustable curved bracket connecting the reference element and the cannulated guide whereby the footprint target tip can be positioned on the bone from a posterior position to the bone and the cannulated guide can be positioned anterior to the bone.

In one example embodiment of the guide assembly for locating a tunnel position on an attachment surface of a bone for a joint ligament reconstruction comprises a reference element having a footprint target tip having a plurality of apertures, at least one cannulated guide having a longitudinal bore defining a longitudinal bore axis and a bracket to align the longitudinal bore of the cannulated guide relative to the plurality of apertures whereby the reference element can be positioned from a anterior position to the bone. In some embodiments, the bracket is an adjustable curved bracket connecting the reference element and the cannulated guide whereby the footprint target tip can be positioned on the bone from an anterior position to the bone and the cannulated guide can be positioned anterior to the bone.

In some embodiments, the plurality of apertures comprises at least a first, second and third aperture, each aperture being generally aligned serially on the footprint target tip with the second aperture being positioned between the first and the third aperture, each aperture having a center with each center offset by a predetermined separation and the predetermined separation being within an inclusive range of about two (2) to six (6) millimeters between the second center and the first and between the second center and the third center whereby the alignment of the second aperture and the cannulated guide locates a tibial bone tunnel position for a single bundle ACL replacement procedure and the alignment of the first and the third aperture and the cannulated guide locates the tibial bone tunnel positions for a multi bundle ACL replacement procedure.

In some embodiments, the footprint target tip comprises a flattened lower surface configured to nest with a tibial plateau of an intercondylar notch of the bone for an ACL replacement procedure.

In some embodiments, the footprint target tip comprises a flattened lower surface configured to nest with a roof surface of a femoral intercondylar notch of the bone for an ACL replacement procedure.

In some embodiments, the footprint target tip comprises a flattened lower surface configured to nest with a roof surface of a femoral intercondylar notch of the bone for a PCL replacement procedure.

In some embodiments, the plurality of apertures comprises at least a first, second and third aperture, each aperture being generally aligned serially on the footprint target tip with the second aperture being positioned between the first and the third aperture, each aperture having a center and each center offset by a predetermined separation and the predetermined separation being within an inclusive range of about two (2) to six (6) millimeters between a second center and a first and a third center whereby the alignment of the second aperture and the cannulated guide locates a tibial bone tunnel position for a single bundle ACL replacement procedure and the alignment of the first and the third aperture and the cannulated guide locates the tibial bone tunnel positions for a multi bundle ACL replacement procedure.

In one example embodiment of a method for locating a tunnel position on an attachment surface of a bone for a joint ligament reconstruction, the method comprises the steps of positioning a reference element having a footprint target tip posterior on a tibia through a posterior entry portal to locate a tibia tunnel exit point and positioning a distal end of at least one cannulated guide on the tibia to locate at least one tibial tunnel position for at least one tibial bone tunnel. In some embodiments, the step of positioning the reference elements comprises positioning the footprint target tip having a first aperture for locating an anteromedial bundle tunnel position and a second aperture for locating a posterolateral bundle tunnel position and the step of positioning the distal end of at least one cannulated guide comprises positioning the distal end of a first cannulated guide for locating an anteromedial bundle tunnel position and a second cannulated guide for locating a posterolateral bundle tunnel position whereby the at least one tibial tunnel position comprises an anteromedial bundle tunnel position and a posterolateral bundle tunnel position. In some embodiments, the reference element is positioned from an anterior entry portal.

### BRIEF DESCRIPTION OF THE SEVERAL VIEWS OF THE DRAWINGS

In order that the manner in which the above-recited and other advantages and features of the invention are obtained, a more particular description of the invention briefly described above will be rendered by reference to specific embodiments thereof which are illustrated in the appended drawings. Understanding that these drawings depict only typical embodiments of the invention and are not therefore to be considered to be limiting of its scope, the invention will be described and explained with additional specificity and detail through the use of the accompanying drawings in which:

FIG. 1 illustrates a top perspective view of one embodiment of the ligament reconstruction guide assembly;

FIG. 2 illustrates a top perspective view of a partially exploded view of one embodiment of the reconstruction guide assembly;

FIG. 3 illustrates a top perspective view of one embodiment of the ligament reconstruction guide assembly showing the alignment of a cannulated guide and the reference element;

FIG. 4 illustrates a side view of one embodiment of the ligament reconstruction guide assembly;

FIG. 5A-5B illustrates a perspective view of embodiments of individual components of a ligament reconstruction guide assembly;

FIG. 6 illustrates a process diagram of one embodiment of the methods of the invention;

FIG. 7 illustrates a side view of one embodiment of the ligament reconstruction guide assembly positioned for use;

FIG. 8 illustrates a side, partially cut-away view of the general positioning of the attachment site of a posterior cruciate ligament (PCL) on a right femur;

FIG. 9A illustrates a top view of a right tibia showing the general positioning of the attachment site of an ACL.

FIG. 9B illustrates a side, partially cut-away view of the general positioning of the attachment site of an ACL;

FIG. 10A-10D shows perspective views of embodiments of footprint target tips for use with multiple ligament reconstruction procedures primarily from a posterior entry;

FIG. 11 is a perspective view of one embodiment of a guide assembly with multiple cannulated guides and a target tip for posterior entry;

FIG. 12 is a perspective view of one embodiment of a guide assembly with multiple cannulated guides and a target tip for posterior entry;

FIG. 13A-13D shows perspective views of embodiments of footprint target tips for use with multiple ligament reconstruction procedures primarily from an anterior entry;

FIG. 14A shows a top, partially cut-away view of one embodiment of a guide assembly with a footprint target tip and multiple cannulated guides;

FIG. 14B shows a top, partially cut-away view of another embodiment of a guide assembly with a footprint target tip and multiple cannulated guides;

FIG. 15A shows a perspective view of a tibia and femur showing an example of the alignment angles of the tibia and femur bone tunnels for an ACL reconstruction compared to the vertical axis of the tibia; and

FIG. 15B shows a top view of a femur showing an example of the alignment angles of the tibia and femur bone tunnels for ACL and PCL reconstruction in a plane perpendicular to the vertical axis of the tibia.

### DETAILED DESCRIPTION OF THE INVENTION

A ligament reconstruction guide assembly and methods for use will now be described in detail with reference to the accompanying drawings. It will be appreciated that although embodiments are described for use with ligament reconstruction, it is understood that the methods and systems described can be use for use in similar medical procedures where the positioning of tunnels, holes or other portals must be carefully placed. Notwithstanding the specific example embodiments set forth below, all such variations and modifications that would be envisioned by one of ordinary skill in the art are intended to fall within the scope of this disclosure.

Embodiments of this invention recognize the benefits that are available by positioning the ligament reconstruction guide assembly from either posterior or anterior positions to the knee. By positioning the target tip of the assembly from a posterior position, it has been found that proper tunnel locations can be made more easily on the surgical table which is an environment where space and time to act are limited. Positioning the target tip of the assembly from an anterior position can also provide accurate tunnel positioning for certain reconstruction procedures.

Embodiments of this invention also relate in part to advancing the art of single and double-bundle reconstruction surgery by recognizing that it is important to locate the bundle attachment sites in order to be able to properly guide the surgeon to create the correct tunnel sites. Embodiments of this invention provide several different systems for accurately locating the bundle attachment points in single and double-bundle procedures in ACL and PCL reconstructions. In particular, these systems enable accurate positioning of ACL and PCL attachment sites from a posterior or anterior position as well as attachment sites for either single of double bundle procedures. This provides a greater degree of flexibility, accuracy and reproducibility than with currently available instruments.

### Ligament Reconstruction Guide Assembly for Posterior Entry:

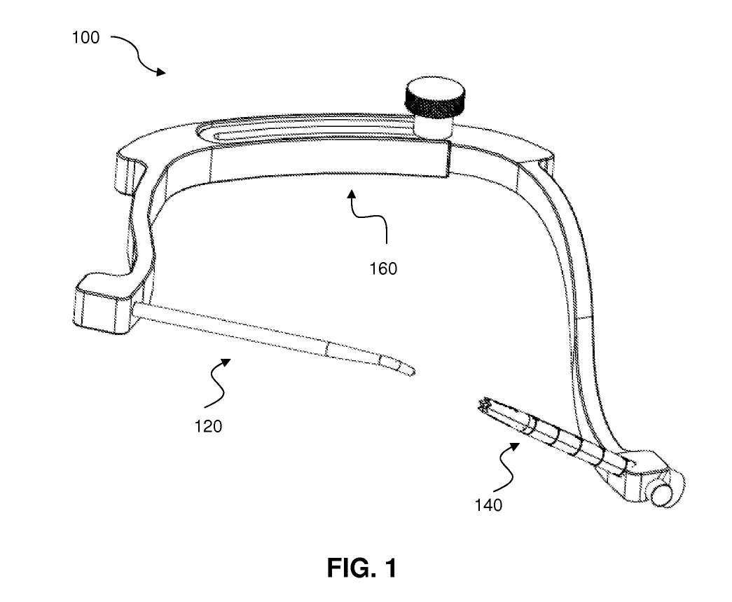

One embodiment of the ligament reconstruction guide assembly 100, as shown in FIG. 1, comprises a bracket 160, a cannulated guide 140 and a reference element 120. These assembly elements are combined and shaped in a manner that permit a surgeon to carefully position a bone tunnel in a ligament reconstruction procedure extending from a posterior position to the knee to an anterior position relative to the knee. The bone tunnel is positioned by the reference element 120 posterior to the knee to an anterior position relative to the knee positioned by the cannulated guide 140. The cannulated guide 140 guides a guide wire or drill to create a tunnel aligned with the reference element 120. This combination and shape allows proper positioning and reduces the possibility of wounds to the posterior elements of the knee.

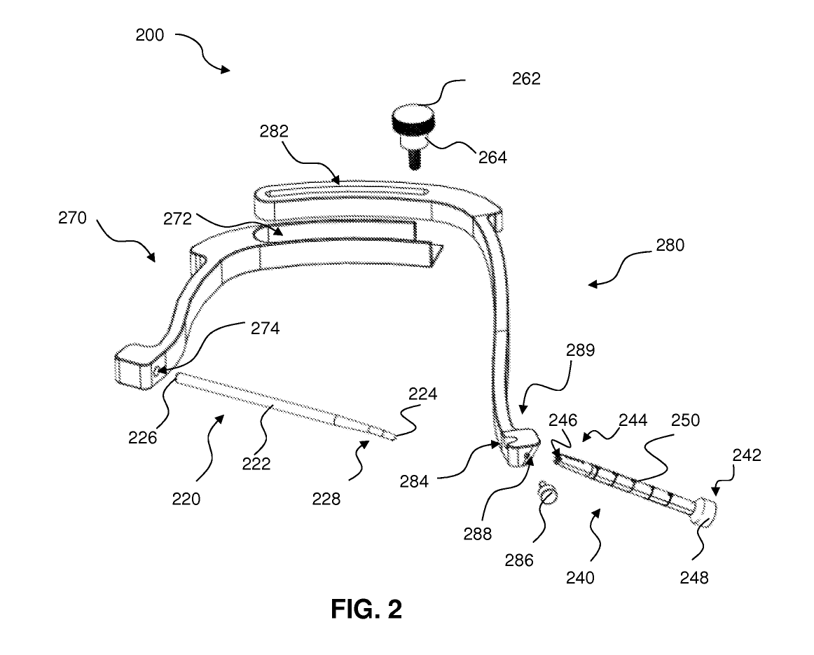

One embodiment of the bracket 160, as shown in FIG. 1, comprises a rigid U-shaped bracket connecting the cannulated guide 140 and the reference element 120. The bracket 160 made of suitable rigid surgical materials such as, but not limited to titanium, stainless steel, nitinol, metal alloys, plastics or other suitable synthetic materials. The bracket is an adjustable curved bracket having an adjustment means to allow the bracket to move through multiple size arches and once the suitable arch is obtained, the arched bracket can be secured into that shape. In the embodiment of FIG. 2, the adjustment means comprises a rod bracket arm 270 and a guide bracket arm 280 that are slidably connected by a cooperating channel 272 that cooperatingly receives the profile of the guide bracket arm 280. The shape of the channel 272 and the bracket arm profile cooperate such that as adjustments are made to the two arms, a proper relationship is maintained between the reference element 220 and the cannulated guide 240. This proper relationship is one where a central radial point about the reference element is created and maintained within the arched bracket even when adjusted. Suitable securing means to secure the bracket arms comprises means such as a nut and bolt, pins, clips or other securing means.

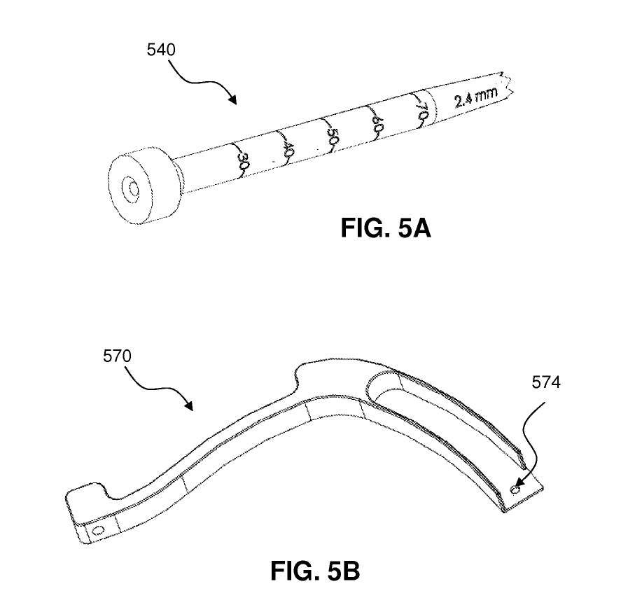

FIG. 2 also shows a threaded knob 262 as the means to secure the two bracket arms. In this embodiment, the threaded knob 262 is received in a mating threaded aperture (see 574 in FIG. 5A) in the rod bracket arm 270. This knob 262 is also received through a slot 282 in the guide bracket arm 280. When the knob 262 is threaded into the threaded aperture, the knob tightens on the guide bracket arm 280, a widened width 264 of the knob puts a force on the guide bracket arm 280 such that it and the rod bracket arm 270 are frictionally engaged and secured by the threaded knob 262. By unthreading the knob 262 from the bracket arms, the two bracket arms can be repositioned and they can also be frictionally engaged in this or other positions by tightening the threads on the threaded knob 262.

The rod bracket arm 270 has a connection means in its distal end to receive and connect to the distal end of the reference element. In FIG. 2, this means allows for proper alignment of the reference element and its components when the reference element is connected and ensures the proper relationship between the reference element 220 and the cannulated guide 240. In the embodiment shown, and not for limitation, the connection means is a recess 274 that allows the reference element proximal end 226 to be rigidly connected by a means such as welding. Other connection means such as mated treads, nuts and bolts, pins, clips or other connecting means, even removable means, are contemplated.

The guide bracket arm 280 has a connection means in its distal end to receive and connect to the cannulated guide 240. This connection means is positioned to allow proper alignment of the cannulated guide 240 with respect to the reference element 220. In the embodiment shown in FIG. 2, and not for limitation, the connection means comprises a bore 284 that allows the cannulated guide 240 to be slidably received in the distal end of the guide bracket arm 280. The connection means also includes a guide screw 286 that is received in a guide screw bore 288 to frictionally secure the cannulated guide 240 within the bore 284. Other connection means such as mated treads, nuts and bolts, pins, clips or other connecting means are contemplated.

The bore 284 in the guide bracket arm 280 is also sized to receive other elements used in ligament reconstruction procedures. These other elements include but are not limited to cannulated reamers, coring drill bits, guide pins, collars and other similar instruments used in creating bone tunnels. The bore 284 has a longitudinal axis that aligns with the longitudinal axis of the cannulated guide 240 when received and other elements to ensure proper alignment of these elements with the reference element 220. The internal size of the bore 284 is also sized to closely fit the outside dimension of the received elements to minimize movement in the bore and therefore the deviation of their longitudinal axes.

One embodiment of the reference element 220 is an elongated rigid rod 222 having a target tip 224 at its distal end. The reference element 220 is attached at its proximal end 226 to the free distal end of the rod bracket arm 270. The target tip 224 is positioned inside the radial opening of the curve of the bracket so that it is generally positioned at the central radial point of the arched bracket. Although not necessary, the embodiment in FIG. 1, the reference element 220 has a bend 228 in the rod 222 that helps the assembly maneuver the target tip 224 during use and position the target tip when positioned from a posterior medial portal during surgery.

The reference element 220 is made of a rigid surgical material such as but not limited to but not limited to titanium, stainless steel, nitinol, metal alloys, plastics or other suitable synthetic materials. The length and diameter of the reference element is sized to provide a small profile in the patients' body when used. Suitable dimensions for illustration, and not for limitation include the reference element having a length ranging from about 4 to 5 inches and more preferably about 4.5 inches and a diameter ranging from about 0.17 to 0.2 inches and more preferably 0.187 to 0.188 inches tapering to a point at its distal tip. For reference elements with a bend, the bend can be any angle that helps the user position the distal tip of the element. For illustration purposes and not for limitation, for embodiments of the reference elements with a bend, the inner angle of the bend in the reference element can be about 100-170 degrees and in one preferred embodiment, about 130 degrees.

In an embodiment of the reference element, a shield is also provided to help prevent the insertion of elements through the cannulated guide beyond the shield. The shield can be an enlarged portion of the target tip or is may be a portion of the reference element separate from the target tip such as a generally flat plate. It is also contemplated that embodiments of the assembly provide for the target tip to be positioned relative to a radial center of the assembly, and this shield may be placed at that radial center. These types of embodiments will allow positioning of the assembly with the target tip while the cannulated guide allows guide pins to be inserted and the shield prevents the guide pin from damaging tissues in the knee beyond the shield.

Referring to the embodiment in FIG. 2, the reference element 220 is connected to the rod bracket arm 270. This connection may be permanent to ensure proper alignment of the target tip 224 with the cannulated guide 240. Removable connections are also possible if they can maintain the proper alignment of the target tip 224 with the cannulated guide 240 as discussed below.

The cannulated guide 240 is a rigid elongated cylinder with proximal end 242, a distal end 244 and a longitudinal bore axis running down a longitudinal bore 246 of the cannulated guide. The distal end 244 of the guide 240 can be, but need not be serrated. Serrated edges help secure the cannulated guide on bone during surgery and can help bore into the bone when necessary. The proximal end 242 of the guide 240 can be, but need not be an enlarged portion 248. The longitudinal bore 246 extends through the length of the cannulated guide 240 and is large enough to allow surgical tools such as but not limited to guide pins, drill bits and other tools to be received through the hollow center. The length and diameter of the cannulated guide 240 are sized to provide a suitable length such that the user can position the distal end 244 of the guide on or in the patient's bone while also safely inserting a guide wire or guide pin to position the tunnel. Diameters of the longitudinal bore are those typical for surgical cannulated guides used in orthopedic procedures. Other dimensions of the cannulated guide 240 are those typical for surgical cannulated guides used in orthopedic procedures. Preferably, the cannulated guide 240 is constructed of stainless steel, although it is appreciated that any suitable surgical material may be used.

For illustration, and not for limitation, one embodiment of the cannulated guide 240 includes a diameter of the longitudinal bore 246 of about 0.095-0.099 inches. For illustration and not for limitation, one embodiment of the cannulated guide 240 has an overall length of about 3.2-3.4 inches and an outer diameter of about 0.25 inches tapering towards its distal end 244. As shown in the embodiment of FIG. 1, the enlarged portion 248 has an outer diameter or about 0.5 inches.

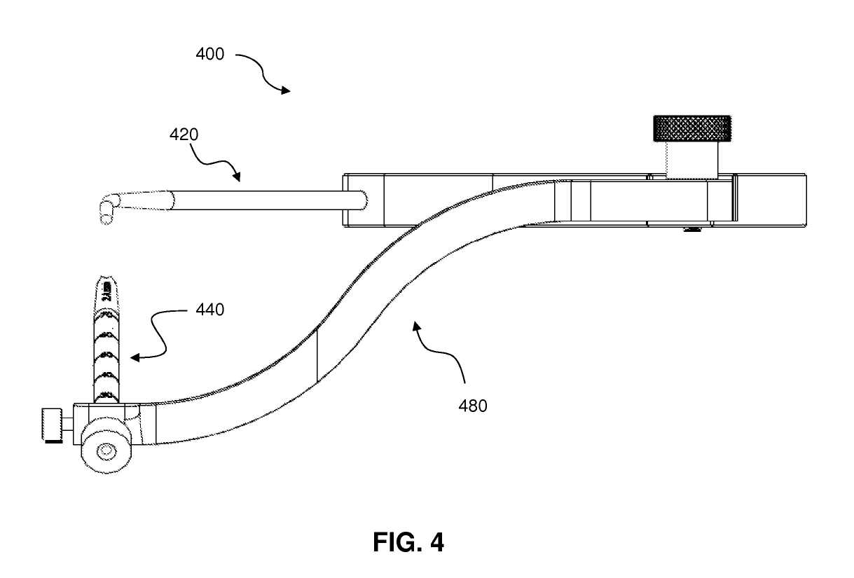

The cannulated guide 240 can be graduated with a series of calibrated markings 250 thereon. In the embodiment shown in FIG. 4, the markings 250 are in 10 mm increments and are used to determine the placement and positioning of the guide pin and the cannulated guide.

The cannulated guide 240 is removably attached to the free distal end 289 of the guide bracket arm 280 by the bracket connection means. The distal end 244 of the cannulated guide 240 is positioned towards the inner center of the bracket arch and aligned relative to the reference element 220 of the assembly 200. The attachment of the cannulated guide 240 to the guide bracket arm 280 is such that it will always allow the extended longitudinal axis of the longitudinal bore 246 to cross the radial center of the arched bracket. This extended longitudinal axis means the center line of the longitudinal bore 246 of the cannulated guide 240, whether directly within the length of the cannulated guide 240 or extending beyond its length.

One means of removably connecting the cannulated guide 240 to the guide bracket arm 280 is to have the guide fit through a bore 284 in the free end of the bracket and have a threaded guide screw 286 frictionally hold the cannulated guide 240 in place. Other attachment means are contemplated such as clips and other frictional attachment methods.

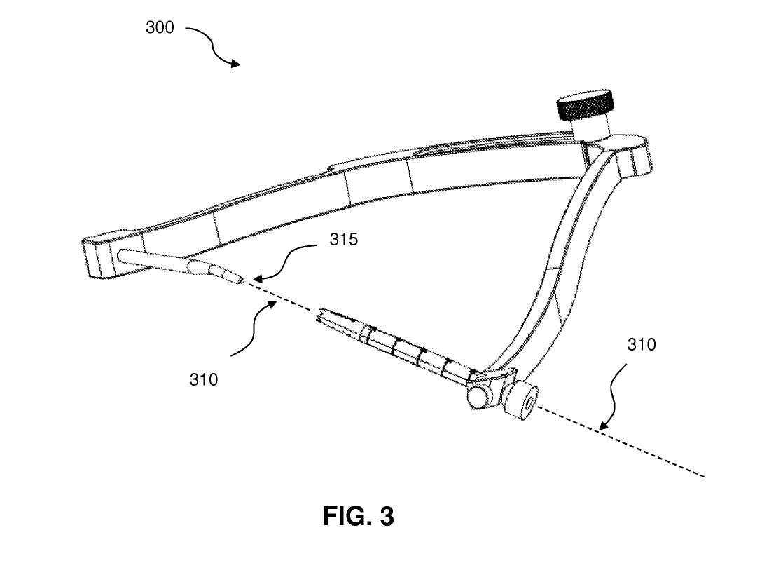

As discussed above, the shape and design of the assembly 200 provides an alignment means to ensure a proper relationship between the assembly elements, in particular a means to align the cannulated guide relative to the reference element. As shown in FIG. 3, when assembled, embodiments of the assembly 300 generally have a radial center. In the embodiment of FIG. 3, the radial center is the point 315 about which the elements cooperate to ensure the extended longitudinal axis of the cannulated guide is properly aligned with the reference element. The dotted line 310 represents the proper alignment of the extended longitudinal axis of the cannulated guide in relation to the target tip of the reference element. In some embodiments, the longitudinal axis is aligned to intersect directly with the tip of the reference elements. In this relationship, the target tip of the reference element is the central point of a circle and the longitudinal axis of the cannulated guide rotates about this point radially as the bracket arms are adjusted. This alignment allows the cannulated guide to guide a straight tool, such as a guide pin, a drill or a reamer, through the cannulated guide hollow center and create a bone tunnel toward the target tip. It is also contemplated that the radial center be another reference point, relative to the target tip but different from the target tip such that a tunnel can be created to that reference point. It is also understood and contemplated that similar relationships between a reference point and the longitudinal axis of the cannulated guide can be used, such as, but not limited to relationships that can be obtained through other geometric relationships such as focal points and parabolic shapes.

When assembled, the assembly also defines a gap between the reference point and the distal end of the cannulated guide. This gap is typically, but not necessarily, a gap ranging through the typical lengths of a tibial bone tunnel for a ligament reconstruction procedure. For illustration and not for limitation, one embodiment of the assembly has a gap range of about 1 to 1.5 inches or preferably about 1.2 inches. This gap can be adjusted by adjusting the position of the cannulated guide in the guide bracket arm bore.

In the embodiment illustrated in FIG. 1, the general shape of the assembly 100 is generally in a single plane. This shape allows the user to position the assembly 100 during a PCL reconstruction procedure while avoiding the obstruction caused by the patient's femur and upper leg. In this first two-dimensional plane, the bracket is curved about the radial center. In this embodiment, the assembly is also curved about a second two-dimensional plane as shown in FIG. 4 to help the user position the assembly 400 around the patient's knee. About this second plane, the guide bracket arm 480 has a s-curve shape. This s-curve can generally be at many angles that ensure proper element alignment. For illustration and not limitation, the s-curve in FIG. 4 is defined by an initial curve of about 45 degrees away from the first two-dimensional plane and a second curve of about 45 degrees back toward the first two-dimensional plane. As shown in FIG. 4, the bore 484 (not shown) in the guide bracket arm 480 is also slanted to allow the cannulated guide 440 to be properly aligned with the reference element 420. Although this embodiment has a s-curve shape and a slanted bore, it is understood that embodiments are contemplated without the s-curve or slanted bore that still keep the proper relationship between the cannulated guide and the reference element.

The embodiment shown in FIG. 5 illustrates one embodiment of the cannulated guide 540, the rod bracket arm 570 and the threaded aperture 574.

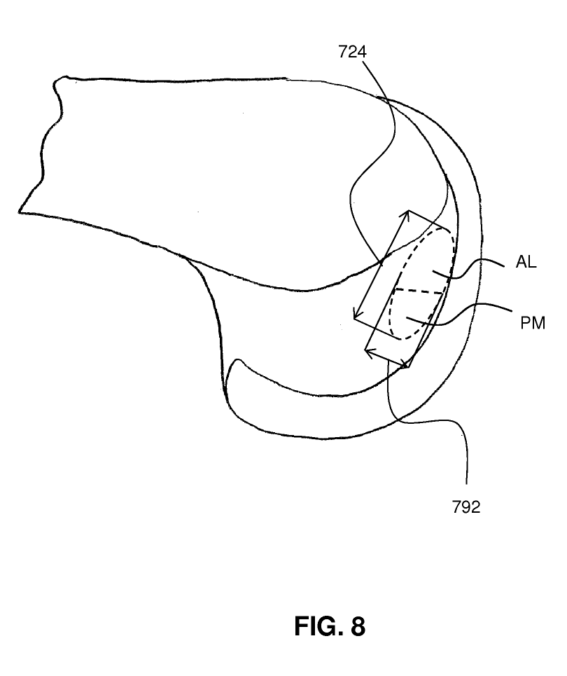

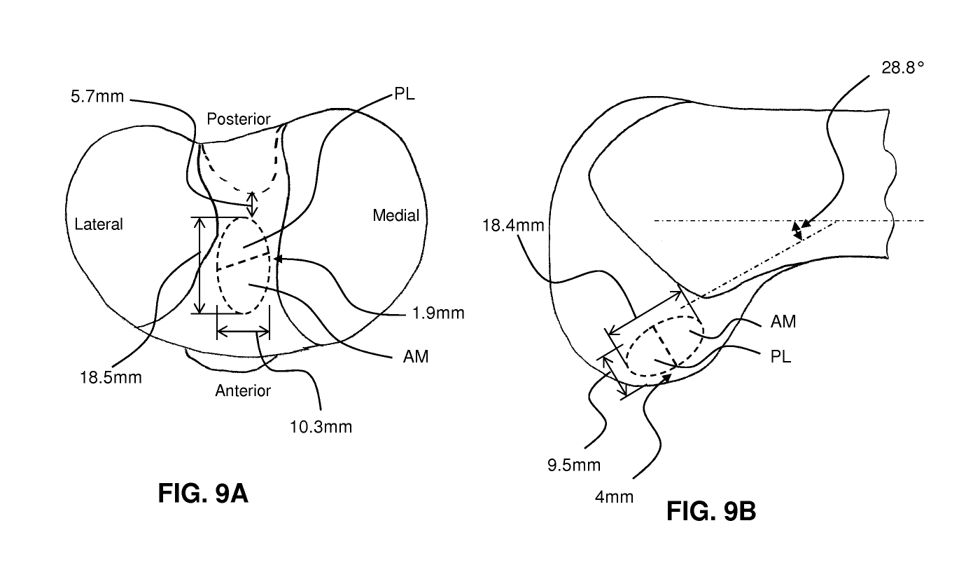

Using the features of the guide assembly, guide systems can be implemented that further assist the surgeon during ligament reconstruction. Guide systems and assemblies can be configured to take advantage of defined anatomical placement sites for single or multi-bundle ligaments. The placement location of PCL ligaments on the femur (right) are shown as the dotted circle in FIG. 8 and the placement of ACL ligaments are shown as the dotted circles in FIGS. 9A-9B for the tibia (right) and the femur (right) respectively. For double-bundle procedures, these placement locations represent locations for different bundle components such as the anteromedial (AM) ligament and the posterolateral (PL) ligament in ACL procedures and posteromedial (PM) ligament and the anterolateral (AL) ligament in PCL procedures. Although specific dimensions of these areas are identified in FIGS. 8, 9A and 9B, it is understood that the dimensions of these areas vary one each patient based on many factors such as the sex, age and bone structure of the patient. Similar descriptions of the anatomy of ACL and PCL attachment sites, as utilized in embodiments described herein, are included in U.S. Pat. Pub. No. 2009/0171355 A1 to Andrew Amis et al, published Jul. 2, 2009 the entire contents of which are herein incorporated by reference.

FIG. 9A shows the attachment site placement of the tunnels in the tibial plateau for an ACL reconstruction procedure. The measurements shown are general “mean” values from different published studies well known in the art. The attachment site can be used to define the attachment site for a single bundle procedure or a double bundle procedure. For a double-bundle ACL procedure, in general, the attachment site comprises separate tunnel attachment site locations or separate sections on one tunnel position for both the AM bundle and the PL bundle.

Using the example positionings shown in FIG. 9A, several methods can be used to define the position of either the whole ACL attachment to the tibia or, as shown by way of example, the AM bundle or the PL bundle as are commonly defined in double-bundle ligament ACL reconstruction procedures. The posterior tibial trans-condylar axis, which is the line passing through the most posterior points on the tibial plateau may be used as the datum from which to define the ACL position in an anterior direction. As shown, this is the example 5.7 mm measurement. Alternatively, or in addition, the ACL may be located in relation to an anterior edge of the tibial plateau by measuring a distance posteriorly from this edge. Another method is to locate the ACL by an anterior distance from the transverse inter-spinous ridge. The medial-lateral position may be defined in relation to the overall medial lateral width of the tibial plateau, as a percentage of this width from the medial edge, for example. Alternatively, the ACL attachment position may be located in relation to landmarks within the knee, for example by medial-lateral measurement from the medial spinous process. As shown, this is the example 1.9 mm measurement. Because it is difficult to locate the summit of the process, it is preferable to measure distances laterally from its steep lateral face. Two dimensional, anterior-posterior and medial-lateral, measurements are helpful to define the ACL attachment position on the tibial plateau.

FIG. 9B illustrates the attachment site placement of PL and AM tunnels in the femur for a double bundle ACL reconstruction procedure. As with FIG. 9A, the measurements shown illustrates mean values of measurements and the attachment site can be used for both single and double bundle reconstruction/replacement procedures. FIG. 9B illustrates how the geometry of the femur (right) may be used to locate the attachment location of a femoral tunnel for an ACL replacement procedure. Landmarks on the femur such as the transverse ridge of the femur, the lateral intercondylar ridge, the lateral bifurcate ridge and the lower surface of the femur may be used as reference points to locate the attachment site.

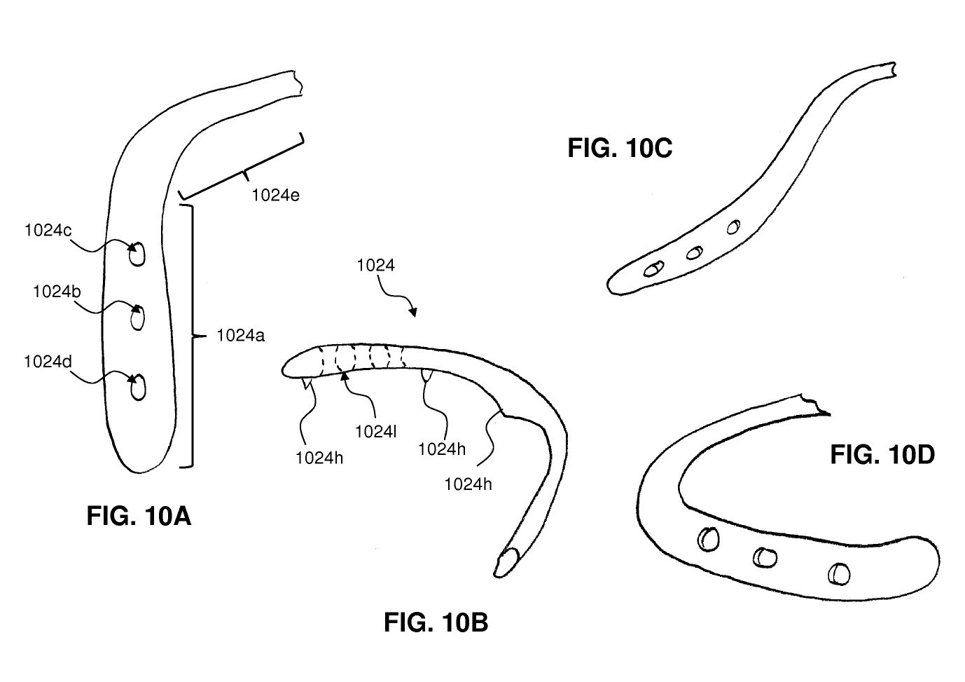

In embodiments of the reference element of the guide assembly, a footprint target tip can be used which has guide recesses or apertures that help ensure the position of guide elements like a guide wire through the attachment site. For example, as shown in FIGS. 10A-10D, the footprint target tips 1024 comprise an elongated tip portion 1024a including multiple apertures to serve as receiving guides for one or more guide pins that when being generally positioned over the attachment site, they will be used to assist guiding bone tunnels positioned by cannulated guides in either single or double bundle procedures. One embodiment of the target tip is an element with three each, 2 mm wide apertures located serially along the length of the tip. The central aperture 1024b is generally in the middle of the guide. The two outer apertures 1024c and 1024d are generally placed to center the attachment sites for AM and PL bundles in a multiple bundle reconstruction procedure. For example, the apertures can be centered on the width of the guide and are generally centered on a first and second half of the guide divided along its length. For example, and not for limitation, the two outer apertures 1024c and 1024d will be located about 5 mm from either end of a 20 mm long footprint target tip. In general, embodiments of the footprint target tip for posterior entry are narrow to ease entry. These apertures can be more finely positioned, such as being off-centered to more accurately reflect the anatomy of the specific ligament it is being used with. The tips can also have a neck portion 1024e that can be curved to more easily accommodate the positioning of the target tip with respect to the other guide assembly elements.

Some embodiments are shaped to allow the guide assembly to locate tunnels from an outside-in position. The outside in position for locating the tunnels avoids the need to put the knee in hyper-flexion during surgery, helps protect the joint and minimizes condyle damage.

FIGS. 10A-10D illustrate example embodiments of footprint target tips that can be used with an guide assemblies designed for posterior entry of the footprint target tip. The appropriate footprint target tip is selected based on the native ACL footprint which can vary as much as about 15 mm in length and 5 mm in width. Target tips are selected after measuring the patient's original ACL footprint and are shaped to extend along the entire footprint which means it contacts the medial tibial spine posterior and the posterior edge of the trans-meniscal ligament anterior. It is also shaped to fit below and between the elevated edges of the tibial plateaus medial and lateral. FIG. 10A shows the top view of an embodiment of a footprint target tip 1024 for an ACL ligament attachment site on the tibial plane of the tibia in a posterior entry procedure on a right knee. FIG. 10B illustrates a side view of the embodiment of FIG. 10A. Example dimensions of a tibial ACL footprint target tip for posterior entry of the footprint target tip are shown below where the aperture center separation represent the predetermined separation distance between the center of the apertures:

| Tibial ACL | Number of | | | Aperture |
|---|---|---|---|---|
| Footprint Target | Guide | | | Center |
| Tip | Apertures | Tip Width | Tip Length | Separation |
| Small | 1 | 5 mm | 10 mm | N/A |
| Medium | 3 | 5 mm | 15 mm | 3.75 mm |
| Large | 3 | 6 mm | 20 mm | 5 mm |

FIG. 10C shows an embodiment of a footprint target tip for an ACL ligament attachment site on the femur in a posterior entry procedure on a right knee. Example dimensions of femur ACL footprint target tip are shown below:

| Femur ACL | Number of | | | Aperture |
|---|---|---|---|---|
| Footprint Target | Guide | | | Center |
| Tip | Apertures | Tip Width | Tip Length | Separation |
| Small | 1 | 4 mm | 10 mm | N/A |
| Medium | 3 | 4 mm | 15 mm | 3.75 mm |
| Large | 3 | 5 mm | 20 mm | 5 mm |

And FIG. 10D shows an embodiment of a footprint target tip for a PCL ligament attachment site on a femur in a posterior entry procedure on a right knee. Example dimensions of femur PCL footprint target tip are shown below:

| Femur PCL | Number of | | | Aperture |
|---|---|---|---|---|
| Footprint Target | Guide | | | Center |
| Tip | Apertures | Tip Width | Tip Length | Separation |
| Small | 1 | 4 mm | 10 mm | N/A |
| Medium | 3 | 4 mm | 15 mm | 3.75 mm |
| Large | 3 | 5 mm | 20 mm | 5 mm |

As illustrated, the footprint target tips can have an elongated tip portion 1024a and a curved neck portion 1024e to conform to the shape of the attachment location. This shaping provides a more steady reference point for the surgeon when the tip undersurface is placed on the attachment surface. The shaping also provides the opportunity to move the tip on the attachment surface until it can be referenced by or engaged with physical landmarks near the attachment location. For example, in FIG. 10B, the footprint target tip 1024 has one or more protrusion 1024h on the flattened lower surface 1024l of the tip that can be used to frictionally engage or locate boney landmarks on the tibia. Examples of protrusions can include sharp spikes or they can be smooth protrusions either of which can engage bony reference points such as, but not limited to, the edges of the ligament attachment site, the medial spinous process or the transverse inter-spinous ridge for tibial landmarks. Similarly, protrusions can engage reference points on the femur such as the transverse ridge, the lateral intercondylar ridge, the lateral bifurcate ridge or the lower surface of the femur. The dimensions of the tip may also be shaped to have an edge or a curve of the tip to engage and rest against landmarks. Also, the footprint target tips can be shaped such that they can be specific to a left and right side ligament replacement procedure. For example, for posterior entry, consistent with the description above, the surgeon may prefer to have the guide bracket wrap around the medial side of the knee and the shapes of the embodiments of FIGS. 10A-10D are suitable examples for a procedure on a right knee. It is understood that embodiments may be configured to have the surgeon wrap the guide bracket around either the lateral or medial side of the knee and the curve of the neck portion of the footprint target tip and rod can be made so that they accommodate the tip coming from that side of the knee.

As shown in the examples of FIGS. 10A-10D, each of the tip apertures are positioned to properly locate a single tunnel in a single-bundle reconstruction or two tunnels in a double-bundle procedure. Although single guides with multiple apertures are illustrated, it is contemplated that different size guides may have different sizes with different aperture spacing to represent different sized patients and different procedures. For example, small guides may only have one aperture while medium and large guides may have three apertures to accommodate either a single guide wire through a middle aperture (single bundle procedure) or multiple guide wires through the two outer apertures (double bundle procedure). Embodiments having only two apertures for a double-bundle procedure are also possible. The footprint target tips may be integrated or permanently attached to the reference element or they may be removable elements that can be removed and replaced on a generic reference element.

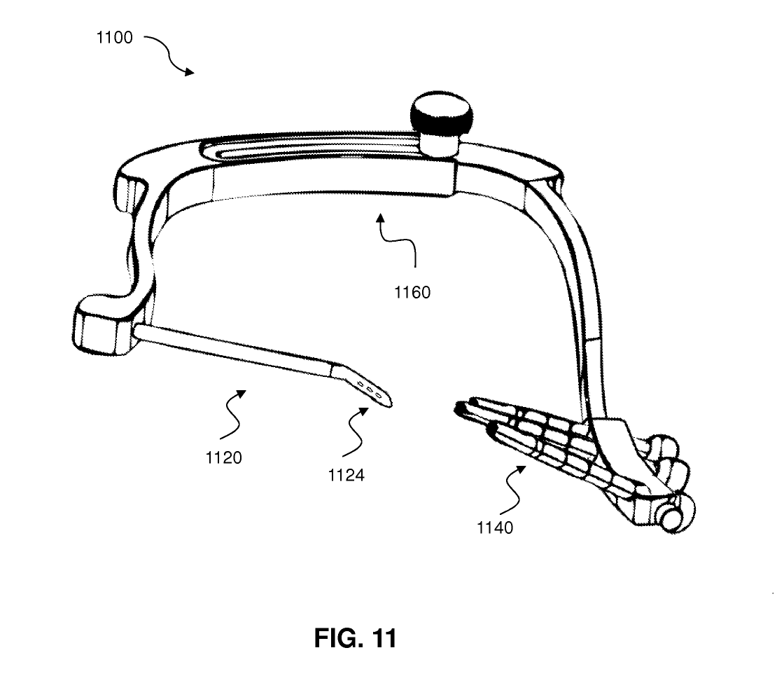

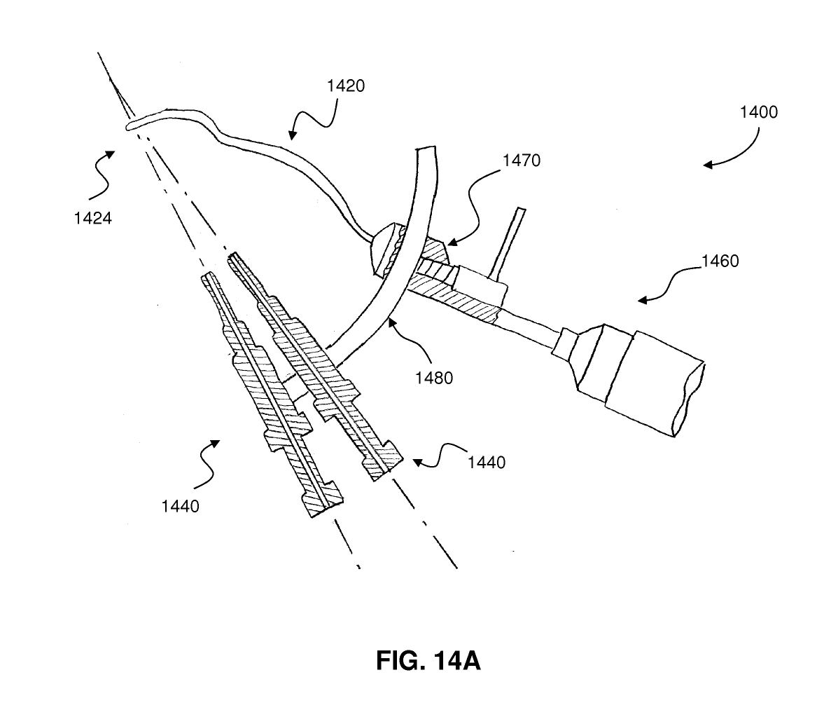

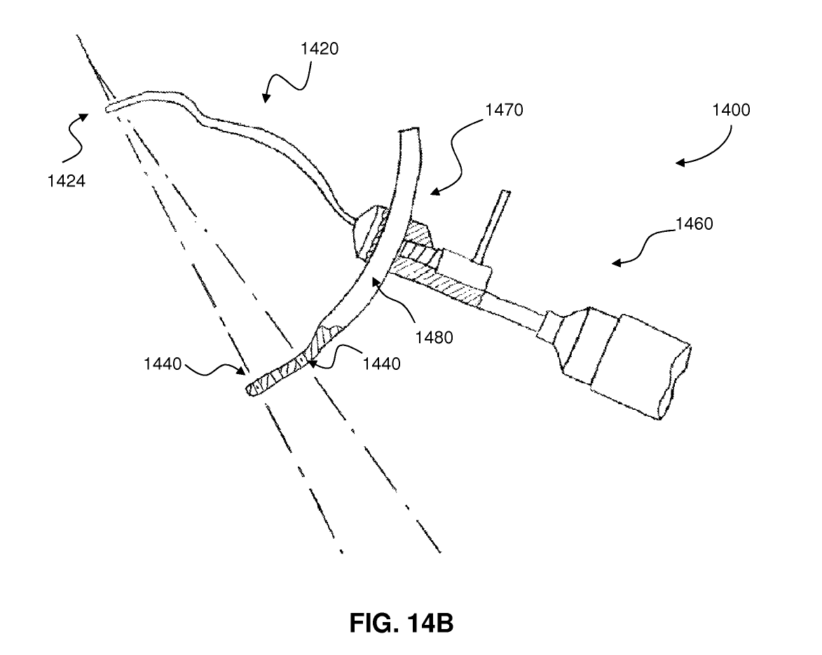

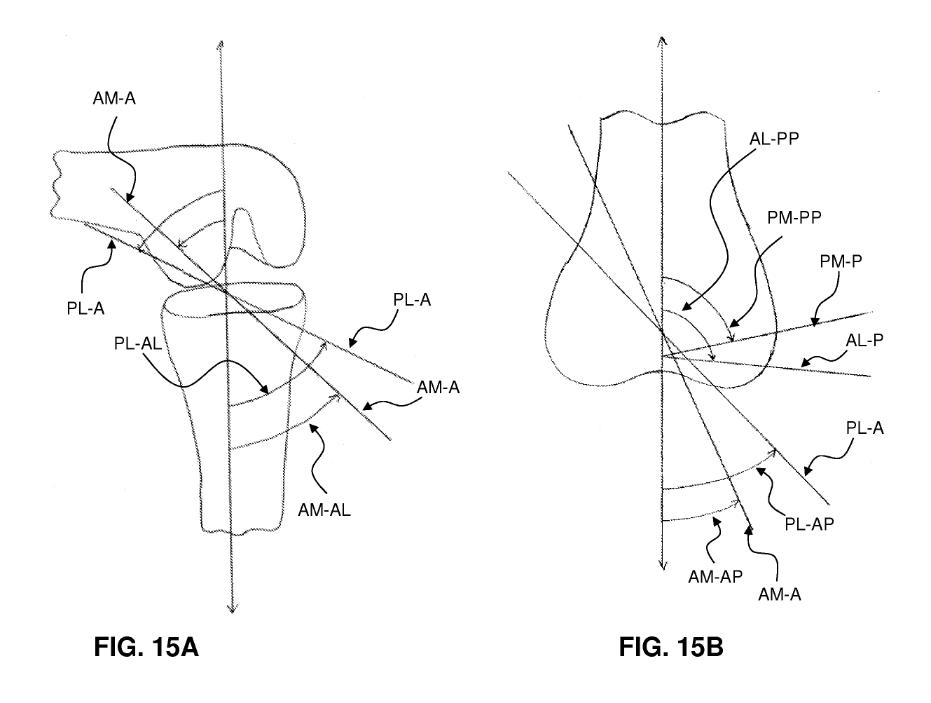

To support single or double-bundle procedures, multiple cannulated guides can be used to guide the creation of the tunnels to receive the bundles. One example of a guide assembly for posterior entry is shown in FIG. 11. This embodiment can be used for posterior entry of a guide for ACL reconstruction at a femur attachment site. Similar to embodiments described above, this embodiment includes a bracket 1160, multiple cannulated guides 1140 and a reference element 1120. This reference element includes a footprint target tip 1124 that may be used to guide the femoral bone tunnel position in a femur for an ACL reconstruction. As with the guide assemblies described above, the bracket allows the cannulated guides to move through different angles. The alignment of the cannulated guides to each other as shown is generally perpendicular to the vertical axis of the tibia and may be aligned with the apertures of the footprint target tip for that procedure. As shown, the bracket allows the guides to be adjusted in the plane generally perpendicular to the vertical axis of the tibia while still staying aligned with apertures of the target tip. The apertures in the footprint target tip are shaped such that they allow for the different angles of the cannulated guides yet they provide a consistent footprint for tunnel placement on the bone. The cannulated guides are configured such that as they are used for guiding tunnel creation and the guides have generally converging angles (see example guide alignment in FIGS. 14A, 14B, 15A and 15B). For the femur tunnel placement, the angles of the guides may create extended axes that converge or cross before extending onto the femur attachment surface. For use with ACL procedures, the angles of the longitudinal axis of an embodiment of three cannulated guides many have a inclusive range generally between about 5 and 20 degrees from each other. In a preferred embodiment, the angular differences between the axes are about 10 degrees from each other.

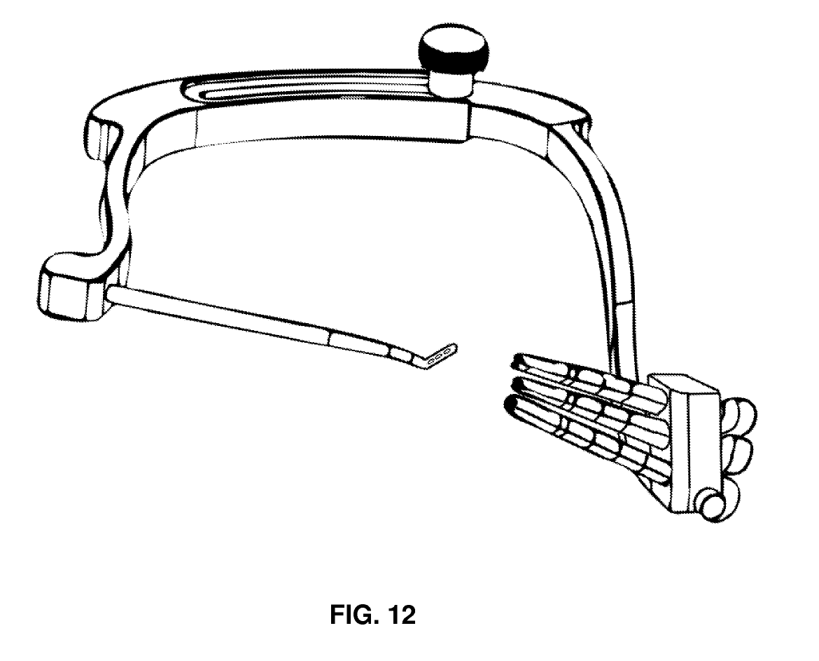

FIG. 12 illustrates another embodiment of a guide system for guiding the creation of tibial tunnels in an ACL reconstruction. In this embodiment, the bracket is adjustable generally in a plane perpendicular to the vertical access of the tibia. It is contemplated that the guide assembly can also be configured to be adjustable along other planes to include planes generally parallel to the vertical axis of the tibia as well as having each cannulated guide be adjustable along the guide. The alignment of the cannulated guides as shown is generally in parallel with the vertical axis of the tibia and are preferably aligned with the apertures of the footprint target tip. In embodiments such as that shown in FIG. 12, for use with the tibial attachment site in an ACL procedure, the alignment of the guides with the footprint target tip allow the angles of the tunnels to be placed such that: the AM tunnel is about 30-60 degrees or in a preferred embodiment about 50 degrees from the vertical axis of the tibia shaft(perpendicular to the horizontal plane of the tibia plateau), the PL bundle tunnel is about 60-80 degrees or in a preferred embodiment 70 degrees from the tibia shaft and a single bundle tunnel is about 50-70 degrees or in a preferred embodiment about 60 degrees from the tibia shaft. In these embodiments, the angles generally converge or cross over each other at a point where the AM and PL ligaments would cross between the femur and the tibia. For a single bundle procedure, example tunnel alignment range may be about 50-80 degrees, inclusive, from the tibia shaft (20 to 50 degrees from the horizontal plane of the tibia plateau) or in a preferred embodiment a range of about 60-70 degrees from the tibia shaft. As is described in the methods below, the tunnel guides for the tibial attachment site may also be extended to locate the femur attachment site.

In some embodiments, rather than having the cannulated guides being generally perpendicular or parallel to the plane of the bracket adjustment, the cannulated guides may be aligned on the bracket with an offset reflecting the specific angles desired for tunnel placement for that specific procedures.

FIG. 15A illustrates examples of the angles described above for a right knee. As shown, the AM-A and PL-A represent example tunnel alignment off of the vertical axis of the tibia. As an example only, the angle of the AM-A tunnel, the AM-AL angle, is about 50 degrees off of the vertical axis and the angle of the PL-A tunnel, the PL-AL angle, is about 70 degrees off of that axis. These angles are examples of angles for a double bundle ACL replacement from the tibia. As shown, these angles may also be extended to locate the femur tunnel when the knee is in flexion.

FIG. 15B illustrates a view over the femur in flexion and down the vertical axis of the tibia. This view shows the perpendicular plane about the tibia and shows example angles for tunnel positions for ACL and PCL procedures. As shown, the AM tunnel placement for the ACL AM bundle, AM-A, starts on the tibia at an angle in the plane perpendicular to the tibia vertical axis, AM-AP, at an inclusive range of about 15-35 degrees medial from the longitudinal axis of the femur shaft or in a preferred embodiment a range of about 20-30 degrees. The PL tunnel placement for the ACL PL bundle, PL-A, is at an angle in the plane perpendicular to the tibia vertical axis, PL-AP, at an inclusive range of about 40-70 degrees medial from the longitudinal axis of the tibia femur or in a preferred embodiment a range of about 50-60 degrees. Geographically, for an ACL procedure, this generally translates to a position of the PL-A tunnel on the tibia starting about 1 cm lower than the start of the AM-A tunnel and just lateral to the edge of the MCL fibers that attach to the tibia.

As shown in FIG. 15B, for a PCL procedure, starting on the roof surface of the femoral intercondylar notch extending medially through the femur, the anteriorlateral (AL) tunnel placement for the PCL AL bundle, AL-P, is aligned at an inclusive range of about a 80-100 degree angle, AL-PP, medial from the longitudinal axis of the femur shaft or in another embodiment a range of about 85-95 degrees. The posteromedial (PM) tunnel placement for the PCL PM bundle, PM-P starts from the roof surface of the femoral intercondylar notch and extends medially at an inclusive range of about a 70-90-degree angle, PM-PP, medial from the longitudinal axis of the femur or in another embodiment a range of about 75-85 degrees.

It is contemplated that the footprint target tips can be removable tips that can be removed and replaced from a common guide system. The tips can be separate removable elements from the reference element or the reference element can be integrated with the target tip and the reference element can be removable to allow multiple shapes of footprint target tips to be used with the guide system. For example, separate tips can be made removable by having a removable connection such as a threaded end that mates with a threaded end of the rod of the reference elements. The tips can be organized in a kit to allow left, right, femur, tibia, ACL and PCL tips to be provided with the guide system. Given the different applications possible for the guide system, the length of the reference element, with or without the target tip, may be different for each application.

It is understood that although multiple cannulated guide assemblies can be used, embodiments that allow a single cannulated guide to move through different angles can also be used for multiple bundle procedures.

It is understood that the assemblies and systems disclosed can be modified so that they can be used in surgeries for either a left or a right knee. In one embodiment, there is both an assembly for use on a left knee and a different assembly for use on a right knee. In these left and right embodiments for use in knee surgeries, the assembly is modified to allow for the reference element insertion from a posterior position such as a posterior medial portal. In these left and right embodiments, footprint target tips can be used that are shaped specifically for use on a left or right knee.

### Ligament Reconstruction Guide Assembly for Anterior Entry:

Referring to one embodiment shown in FIG. 14, a guide assembly can be configured to enable the anterior entry of the footprint target tip to guide the location of bone tunnels in support of an ACL or PCL reconstruction procedure. Although this embodiment illustrates two cannulated guides, consistent with the embodiments discussed above, a single cannulated guide can be used that moves through the range of different angles required for the procedure as well as an assembly with three cannulated guides.

An example of a suitable guide assembly is described in U.S. Pat. No. 5,112,337, Feb. 5, 1991, entitled “VARIABLE ANGLE, SELECTIVE LENGTH TIBIAL DRILL GUIDE” to Lonnie E. Paulos et al. which is herein incorporated by reference in its entirety. For use with embodiments of the present invention, that guide assembly can be modified to have multiple cannulated guides and modified to have a footprint target tip as described above.

As shown in FIG. 14, the guide assembly 1400 comprises a bracket 1460, multiple cannulated guides 1440 and a reference element 1420. This reference element includes a footprint target tip 1424 that may be used to guide tunnels in a ligament reconstruction. The bracket 1460 includes a guide bracket 1480 and a reference element bracket 1470 that allow the cannulated guides to adjust at different angles to the target tip but still maintain their orientation to each other. As shown, the angles of the axis of the cannulated bore in each of a multiple guide embodiment generally converge where ligament bundles would cross. Shown here is a guide for a tibial tunnel for an ACL replacement. For this configuration, the tunnels converge at a point beyond the target tip where the AM and PL bundles would overlap. This configuration allows the tunnels to be properly placed when being created in an “outside-in” manner. As described above, the angle ranges for embodiments should allow a total offset range of about 5 to 60 degrees between the longitudinal axes of the outer guides. A preferred embodiment provides an offset of about 20 degrees between two outer guides and an offset of about 10 degrees between the two outer guides and a third central guide. As described above, the footprint target tips are configured to be aligned with the cannulated guides so that the tip apertures will accept guide pins placed through the cannulated bores at these angles.

If the embodiment similar to FIG. 14 is used for creating inside-out tunnels in the femur, the footprint target tip will allow the longitudinal axis of the guides to converge prior to the footprint target tip and extend to position angled tunnels in the femur.

As described earlier, embodiments can also enable tunnels to be created from an outside in position.

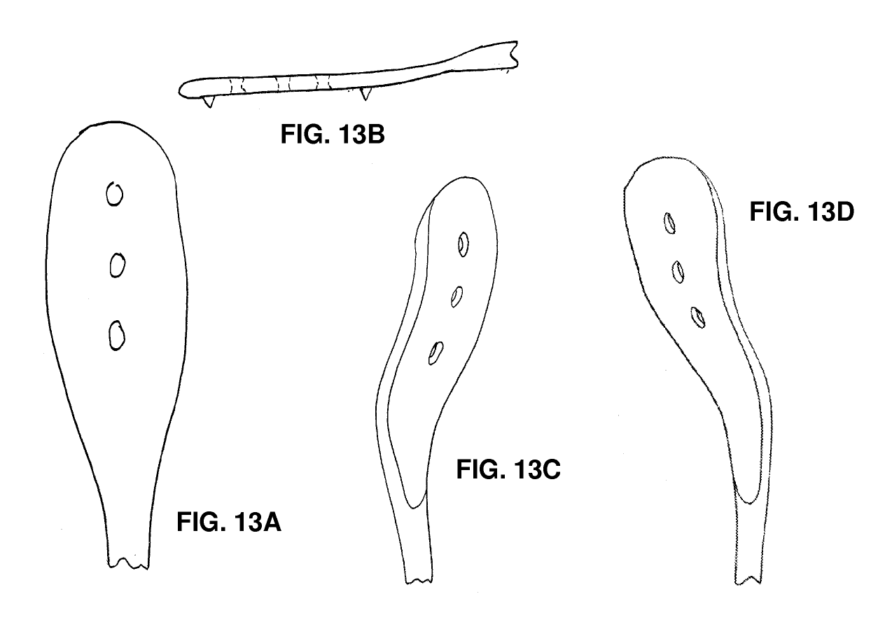

FIG. 13A shows an embodiment of a footprint target tip for an ACL ligament attachment site on the tibial plane of the tibia for use with procedures providing anterior entry of the footprint target tip on a right knee. FIG. 13B illustrates a side view of the embodiment of FIG. 13A. Example dimensions of the anterior entry tibial ACL footprint target tip are shown below:

| Tibial ACL | Number of | | | Aperture |
|---|---|---|---|---|
| Footprint Target | Guide | | | Center |
| Tip | Apertures | Tip Width | Tip Length | Separation |
| Small | 1 | 10 mm | 10 mm | N/A |
| Medium | 3 | 11 mm | 15 mm | 3.75 mm |
| Large | 3 | 12 mm | 20 mm | 5 mm |

Using a guide assembly similar to the one of FIG. 14, a footprint target tip specific to a femur could be used to guide the femur attachment sites. FIG. 13C shows an embodiment of a target tip for an ACL ligament attachment site on the femur during an anterior entry procedure of the footprint target tip on a right knee. Example dimensions of the anterior entry femur ACL footprint target tip are shown below:

| Femur ACL | Number of | | | Aperture |
|---|---|---|---|---|
| Footprint Target | Guide | | | Center |
| Tip | Apertures | Tip Width | Tip Length | Separation |
| Small | 1 | 8 mm | 10 mm | N/A |
| Medium | 3 | 9 mm | 15 mm | 3.75 mm |
| Large | 3 | 10 mm | 20 mm | 5 mm |

FIG. 13D shows an embodiment of a target tip for a PCL ligament attachment site on the femur during an anterior entry procedure of the footprint target tip on a right knee. Example dimensions of the anterior entry femur PCL footprint target tip are shown below:

| Femur PCL | Number of | | | Aperture |
|---|---|---|---|---|
| Footprint Target | Guide | | | Center |
| Tip | Apertures | Tip Width | Tip Length | Separation |
| Small | 1 | 8 mm | 10 mm | N/A |
| Medium | 3 | 9 mm | 15 mm | 3.75 mm |
| Large | 3 | 10 mm | 20 mm | 5 mm |

Although the above example dimensions are listed with a degree of specificity, it is understood that the measurements are meant for illustration purposes and are not meant for limitation. Any dimension that allows the target tip to be inserted into the surgical location without damaging other tissues or interfering with other surgical devices would be suitable. Dimensions that allow for the target tip to engage landmarks on the surface of the tibia and femur are preferred.

### One Embodiment of the Methods of Use of the Ligament Reconstruction Assembly for Posterior Entry:

The following description of one method of use of the ligament reconstruction assembly is to illustrate an embodiment of the methods of use for this assembly and is not intended as a limitation. It should be understood that while this invention is described in connection with particular examples and embodiments, the scope of the invention need not be so limited. Rather, those skilled in the art will appreciate that the following teachings can be used in a much wider variety of applications than the examples specifically mentioned.

One embodiment of the method is for use in arthroscopic posterior cruciate ligament (PCL) reconstruction surgery. In this embodiment, synthetic ligament grafts are provided or harvested and ligaments are reconstructed through well known methods such as those described in U.S. Pat. No. 5,300,077 filed Feb. 23, 1993 and U.S. Pat. No. 6,254,605 filed Nov. 27, 2000 to Howell both of which are herein incorporated by reference in their entirety. Specific to PCL reconstruction surgery, the location of tunnels and ligaments for the tibia and the femur are generally as described in U.S. Pat. No. 4,787,377 filed May 6, 1987 to Laboureau which is herein incorporated by reference in its entirety.

During this arthroscopic procedure, portals for the arthroscope and graft harvesting are made on the patient. Through these portals, the knee is examined by arthroscopic procedures and any observed minor defects or irregularities are taken care of.

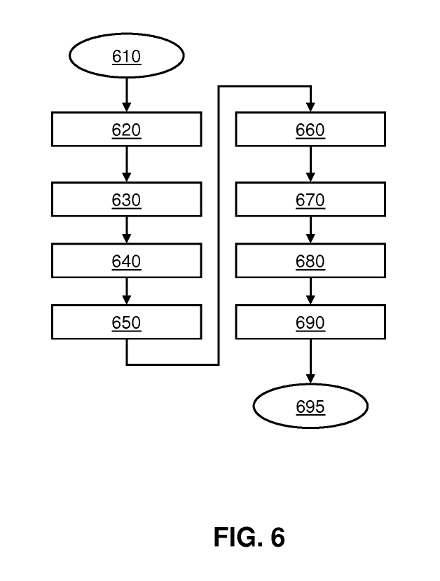

As shown in FIG. 6, one embodiment of the process 600 starts after start 610 with step 620 being positing a reference element of a ligament reconstruction guide assembly. In this embodiment, a ligament reconstruction guide assembly is provided such as shown in FIG. 1 having a bracket, reference element and a cannulated guide. This reference element of the ligament reconstruction guide assembly enters the patient's body through a posterior medial portal from the anterior knee. This entry is made by the target tip of the reference element which can be accurately placed while viewing through the intercondylar notch with the arthroscope. With the arthroscope in this position, it facilitates the surgery and allows the surgeon to clean soft tissues away from the back of the knee without jeopardizing neurovascular structures.

It is also possible to have the reference element and the target tip enter the patient's body from other posterior positions of the knee.

With the target tip of the reference element properly placed, the bracket can be adjusted by adjusting the rod bracket arm relative to the guide bracket arm to allow step 630 which is the positioning the distal end of the cannulated guide on the tibia so that the surgeon can pass a guide wire, drill or reamer through the guide.

Step 640 comprises drilling the transtibial PCL tunnel from the surgeons preferred position on the tibia to exit at the PCL anatomic insertion site as identified by the location of the target tip. With this assembly, during a PCL reconstruction operation, the surgeon can place the distal end of the cannulated guide, and therefore start the bone tunnel, from either an anterior medial or anterior lateral positions on the tibia, depending on the surgeon's preference. The means to keep the alignment of the bone tunnel with the target tip is provided by allowing the surgeon to adjust the shape of the assembly by adjusting the assembly bracket arms and securing them in the new shape with the bracket set screw. The assembly is able to enter from the posterior medial or lateral corners of the knee and can pass a guide pin or guide wire from any anterior position desired (from anterior lateral to anterior medial of the tibial tubercle) and at the same time pass the guide pin or guide wire at the correct inferior to superior angle so as to minimize neuro-vascular damage and reduce edge stress on the graft material. After placement of the entry point of the tunnel, the guide wire, drill or reamer is advanced through the cannulated guide towards the target tip. In one embodiment, the longitudinal axis of the cannulated guide is aligned with the target tip of the rigid rod. In one embodiment, a guide wire is passed by drilling or tapping through the cannulated guide to position the tunnel. If the target tip is placed at a designated exit point for a bone tunnel, the tunnel will be created and have an exit point at that point. In this mode, with the guide wire positioned in the tunnel, the cannulated guide can be removed, a collar can be placed over the guide wire and a cannulated bone boring means can be placed through the guide arm bore and over the collar and guide wire. With the bone boring means, such as but not limited to a coring drill bit, a drill bit or a reamer, aligned over the guide wire, the bone tunnel can be created with a proper alignment to the target tip.

With this posterior entry method, damage to the vascular clump and neurovascular structures of the knee are protected by the small size of the target tip and the surgeon's ability to visualize the exit point with the arthroscope. Because the assembly and guide pin can be passed under direct vision, it is a safe technique to help avoid damage to the neurovascular structures. It is also understood that positioning of the target tip could be done with x-ray equipment as currently used in the art.

Step 650 comprising positioning and drilling the femoral attachment site for the PCL which is visually placed and marked. The femoral lateral cortex is exposed and a bone boring means is utilized to create a small tunnel through the femur.

The surgeon can proceed to the replacement of the PCL of the knee using the medial anterior approach. Step 660 comprises passing the ligament graft through the tibia tunnel. One embodiment of this step is done by means of flexible pin connected to the leading end of the graft. When the flexible pin exits the tibia tunnel posterior, the end of the pins are grasped and pulled through tunnel and then used to position and secure the trailing end of the graft in the tibia tunnel which is step 670. Once positioned in the tibia tunnel, the leading end of the graft is then inserted into the femoral tunnel as step 680. The grafts can be positioned in the tunnel through the use of the flexible pin pulling the graft through the femoral tunnel. Once the leading end is positioned it is secured in the femoral tunnel as step 690.

This embodiment of the method is completed with step 695.

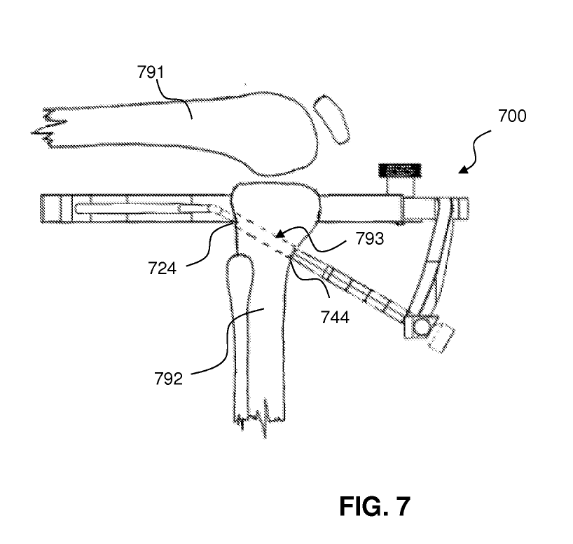

FIG. 7 shows a side view of one embodiment of the reconstruction guide 700 positioned under the femur 791 around the tibia 792 with the tibial bone tunnel 793 show from anterior to posterior aligned between the guide distal end 744 and the target tip 724.

Although the procedures described above include a single tunnel in the tibia and the femur, it is contemplated that the assembly and the methods are just as suitable for a double bundle ligament graft. This would entail multiple tunnels being created in the tibia and the femur to accommodate the multiple graft bundles.

It is also contemplated that although the procedures above include detail using the ligament reconstruction guide assembly to create a tibia bone tunnel, is also understood that embodiments of the guide assembly described herein can be used to create bone tunnels in the femur and other bones from a posterior entry point.

Similar methods may be used to position target tips for locating the tunnel position on the femur for the PCL replacement procedure. In this method, the target tip is inserted posterior and the bottom surface of the footprint target tip is placed against landmarks on the roof surface of the femoral intercondylar notch. Alignment of the tunnels with the cannulated guide can be made by visually aligning the guide medial to the femur and drilling the tunnels utilizing an outside in procedure.

Similar methods may be used to position target tips for ACL replacement procedures from a posterior position. In this method, for placement of the tunnel position on the tibia, the target tip is inserted posterior and the bottom surface of the footprint target tip is placed against landmarks to nest with the tibial plateau of the intercondylar notch. The cannulated guide is positioned visually from an anterior position on the tibia. With the knee in a 90 degree flexed position, the femoral tunnels may be made by extending the tibial tunnels into the femur. Additionally, the placement of the tunnel positions on the femur, the target tip is inserted posterior and the bottom surface of the footprint target tip is placed against landmarks on the roof surface of the femoral intercondylar notch and the alignment of the tunnels can be made by visually aligning the bracket and cannulated guide lateral to the femur and drilling the tunnels utilizing an outside in procedure.

For guide system embodiments with multiple cannulated guides, similar procedures are utilized with guide embodiments such as those shown in FIGS. 11, 12, 14A and 14B. With these multiple cannulated guides, the axes of the guides are aligned with the apertures of the target tip so that multiple tunnels can be located and created. The angles between the cannulated guides can be adjusted depending on the ligament procedure.

### One Embodiment of the Methods of Use of the Ligament Reconstruction Assembly for Anterior Entry:

During an ACL reconstruction, for the tibia tunnels, the footprint target tips are inserted through the anterior medial or accessory medial portal and the scope is in the lateral portal. The bottom surface of the target tip is placed against landmarks to nest with the tibial plateau of the intercondylar notch. Then a tibial incision is made so that the cannulated guide can be pushed against the tibia. For methods using a single bundle, one guide pin is placed at an angle of about 60 degrees from the tibial crest. For methods using a double bundle, one guide pin is placed at about 50 degrees and the other is placed at about 70 degrees. The 50 degree pin is to guide placement of the tunnel for the AM bundle and the 70 degree pin is to guide placement of the tunnel for the PL bundle. Although specific angles are used as examples here, the angle can range at least 10 to 20 degrees on either side of the above angles with satisfactory results.

During an ACL reconstruction, the femur tunnels can be created with the knee at generally 90 degrees of flexion. With the tibia and femur in these positions, the femur tunnels are guided by inserting guide pins through the skin and entering the superior lateral femur from an outside-in position at the same angles to the tibia as described above. An alternative method of locating the femur tunnels is to run one of the femur guide pins from inside-out through a properly aligned tibial tunnel and place the distal hole of a guide assembly on that guide pin and drill the other guide pin from outside-in. For example, the anterior medial femur pin can be placed from inside-out through the anteromedial tibial tunnel and the posterolateral pin can be placed by putting one of the guide assemblies, with the proper angle offset, over the guide pin and use the other guide assembly to position the posterolateral pin from outside -in. Another embodiment is to run one guide pin through the tibial tunnel and use the footprint target tip on a guide assembly to position the other guide pin either from an inside-out or outside-in procedure. Yet another alternative is to use the guide assembly to directly position the femur ACL tunnels using the guide assembly and having an ACL femur specific footprint target tip. Using this embodiment with an outside-in procedure, the knee does not have to be put in hyper-flexion.

For locating the femur attachment sites in a PCL reconstruction procedure, the footprint target tip of the guide assembly can be inserted through the skin from an anterior position with a PCL femur specific footprint target tip. Using this embodiment with an outside-in procedure, the knee does not have to be put in hyper-flexion.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention. Although this invention has been described in the above forms with a certain degree of particularity, it is understood that the present disclosure has been made only by way of example and numerous changes in the details of construction and combination and arrangement of parts may be resorted to without departing from the spirit and scope of the invention.

We claim:

1. A guide assembly for locating a tunnel position on an attachment surface of a bone for a joint ligament reconstruction, said guide assembly comprising:
a reference element having a footprint target tip;
the footprint target tip having a plurality of apertures;
at least one cannulated guide having a longitudinal bore defining a longitudinal bore axis;
a bracket to align the longitudinal bore of the cannulated guide relative to the plurality of apertures; and
the bracket connecting the referent element and the cannulated guide whereby the reference element can be positioned from a posterior position to the bone.

2. The guide assembly of claim 1 wherein the bracket is an adjustable curved bracket connecting the reference element and the cannulated guide whereby the footprint target tip can be positioned on the bone from a posterior position to the bone and the cannulated guide can be positioned anterior to the bone.

3. The guide assembly of claim 1 wherein the reference element further comprises:
a rod having a proximal end connected to a curved bracket and a distal end having the footprint target tip.

4. The guide assembly of claim 1 wherein the at least one cannulated guide comprises a first and second cannulated guide.

5. The guide assembly of claim 1 wherein:
the plurality of apertures comprises at least a first, second and third aperture;
the first, second and third aperture being generally aligned serially on the footprint target tip with the second aperture being positioned between the first and the third aperture;
the first, second and third aperture having a first, second and third center respectively with each center offset by a predetermined separation; and
the predetermined separation being within an inclusive range of about two (2) to six (6) millimeters between the second center and the first and between the second center and the third center whereby the alignment of the second aperture and the cannulated guide locates a tibial bone tunnel position for a single bundle ACL replacement procedure and the alignment of the first and the third aperture and the cannulated guide locates the tibial bone tunnel positions for a multi bundle ACL replacement procedure.

6. The guide assembly of claim 1 wherein the footprint target tip comprises:
a flattened lower surface configured to nest with a tibial plateau of a intercondylar notch of the bone; and
the plurality of apertures comprises at least a first and second aperture, each aperture having a center and each center offset by a predetermined separation whereby the alignment of one of the first and second apertures and the cannulated guide locate a tibial bone tunnel position for a single bundle ACL replacement procedure and the alignment of one of the first and second apertures and the cannulated guide locate the tibial bone tunnel position for a multi bundle ACL replacement procedure.

7. The guide assembly of claim 1 wherein the footprint target tip comprises:
a flattened lower surface configured to nest with a roof surface of a femoral intercondylar notch of the bone; and
the plurality of apertures comprises at least a first and second aperture, each aperture having a center and each center offset by a predetermined separation whereby the alignment of one of the first and second apertures and the cannulated guide locate a femoral bone tunnel position for a single bundle ACL replacement procedure and the alignment of one of the first and second apertures and the cannulated guide locate the femoral bone tunnel position for a multi bundle ACL replacement procedure.

8. The guide assembly of claim 1 wherein the footprint target tip comprises:
the at least one cannulated guide comprises a first cannulated guide for locating an anteromedial bundle tunnel position and a second cannulated guide for locating a posterolateral bundle tunnel position;
the plurality of apertures comprise a first aperture for locating an anteromedial bundle tunnel position and a second aperture for locating a posterolateral bundle tunnel position;
the at least one cannulated guide comprising a first and second cannulated guide;
the first cannulated guide having a first longitudinal bore axis aligning with a center of the first aperture; and
the second cannulated guide having a second longitudinal bore axis aligning with a center of the second aperture whereby the first and second cannulated guide can be positioned from an anterior position to a tibia for locating the tunnel position for an anteromedial bundle and the tunnel position for a posterolateral bundle.

9. The guide assembly of claim 8 wherein a range of an angular difference between the first longitudinal bore axis and the second longitudinal bore axis is an inclusive range of about five (5) to forty (40) degrees.

10. The guide assembly of claim 1 wherein the footprint target tip comprises:
a flattened lower surface configured to nest with a roof surface of a femoral intercondylar notch of the bone; and
the target tip having at least two apertures, each aperture having a center and each center offset by a predetermined separation whereby an alignment of the target tip and the cannulated guide locate a femoral bone tunnel position for a PCL replacement procedure.

11. The guide assembly of claim 1 wherein:
the at least one cannulated guide comprises a first cannulated guide for locating an anterolateral bundle tunnel position and a second cannulated guide for locating a posteromedial bundle tunnel position;
the plurality of apertures comprise a first aperture for locating an anterolateral bundle tunnel position and a second aperture for locating a posteromedial bundle tunnel position;
the at least one cannulated guide comprising a first and second cannulated guide;
the first cannulated guide having a first longitudinal bore axis aligning with a center of the first aperture; and
the second cannulated guide having a second longitudinal bore axis aligning with a center of the second aperture whereby the reference element and the first and second cannulated guide can be positioned to locate an anterolateral bundle tunnel position and the a posteromedial bundle tunnel position for a PCL replacement procedure.

12. The guide assembly of claim 11 wherein a range of an angular difference between the first longitudinal bore axis and the second longitudinal bore axis is an inclusive range of about five (5) to forty (40) degrees.

13. A guide assembly for locating a tunnel position on an attachment surface of a bone for a joint ligament reconstruction, said guide assembly comprising:
a reference element having a footprint target tip;
the footprint target tip having a plurality of apertures;
at least one cannulated guide having a longitudinal bore defining a longitudinal bore axis; and
a bracket to align the longitudinal bore of the cannulated guide relative to the plurality of apertures whereby the reference element can be positioned from a anterior position to the bone.

14. The guide assembly of claim 13 wherein the bracket is an adjustable curved bracket connecting the reference element and the cannulated guide whereby the footprint target tip can be positioned on the bone from a anterior position to the bone and the cannulated guide can be positioned anterior to the bone.

15. The guide assembly of claim 13 wherein the reference element further comprises a rod having a proximal end connected to a curved bracket and a distal end having the footprint target tip.

16. The guide assembly of claim 13 wherein the at least one cannulated guide comprises a first and second cannulated guide.

17. The guide assembly of claim 13 wherein:
the plurality of apertures comprises at least a first, second and third aperture;
the first, second and third aperture being generally aligned serially on the footprint target tip with the second aperture being positioned between the first and the third aperture;
each aperture having a center and each center offset by a predetermined separation; and
the predetermined separation being within an inclusive range of about two (2) to six (6) millimeters between a second center and a first and a third center whereby the alignment of the second aperture and the cannulated guide locates a tibial bone tunnel position for a single bundle ACL replacement procedure and the alignment of the first and the third aperture and the cannulated guide locates the tibial bone tunnel positions for a multi bundle ACL replacement procedure.

18. A method for locating a tunnel position on an attachment surface of a bone for a joint ligament reconstruction comprising the steps of:
positioning a reference element having a footprint target tip posterior on a tibia through a posterior entry portal to locate a tibia tunnel exit point; and
positioning a distal end of at least one cannulated guide on the tibia to locate at least one tibial tunnel position for at least one tibial bone tunnel.

19. The method of claim 18 wherein:
the step of positioning the reference elements comprises positioning the footprint target tip having a first aperture for locating an anteromedial bundle tunnel position and a second aperture for locating a posterolateral bundle tunnel position; and
the step of positioning the distal end of at least one cannulated guide comprises positioning the distal end of a first cannulated guide for locating an anteromedial bundle tunnel position and a second cannulated guide for locating a posterolateral bundle tunnel position whereby the at least one tibial tunnel position comprises an anteromedial bundle tunnel position and a posterolateral bundle tunnel position.
